(19) United States
(12) Patent Application Publication
(10) Pub. No.: US 2009/0234025 A1
(43) Pub. Date: Sep. 17, 2009

(54) Protective Surface Film for a Liquid

(76) Inventor: Graham G. Strachan, Victoria (AU)

Correspondence Address: CHARLES C. MCCLOSKEY, 763 S. NEW BALLAS ROAD STE. 170, ST. LOUIS, MO, 63141, US

(21) Appl. No.: 12/376,246

(22) PCT Filed: Aug. 3, 2007
(86) PCT No.: PCT/AU2007/001094

§ 371 Date: Feb. 18, 2009

(30) Foreign Application Priority Data
Aug. 4, 2006 (AU) 2006904227

(51) Int. Cl. A61K 47/34; C08L 83/04

(52) U.S. Cl. 514/772.3; 524/588

(57) ABSTRACT

A composition and method of protecting the protection of an exposed body of water comprising 5-95 wt % silicone polymer; greater than zero percent to 90 wt % carrier material; and greater than zero −20 wt % surfactant. The method includes the steps of adding the composition to the surface of the water and allowing the composition to form a liquid film across the surface of the water. The film is effective for reducing evaporation of water from the surface.

### FIELD OF THE INVENTION

The present invention relates to a protective surface film for a liquid and in particular stable protective surface films used to cover a large body of water to prevent evaporation.

### BACKGROUND OF THE INVENTION

The need to conserve scarce water supplies is a major environmental issue worldwide. As a result, an increasing level of attention is being directed to various water-saving measures. In particular, the loss of water through evaporation is one area of major concern. Measures to restrict evaporation have included the use of underground drip irrigation, the replacement of open irrigation channels with pipelines and restricting the application of water to times at which evaporation is kept to a minimum.

Several products have also been developed to reduce the evaporation from water bodies by covering the surface of the water, such as shade structures, plastic films and other floating membranes. However, the widespread adoption of such products has been inhibited by the substantial initial capital costs.

Chemical monolayers have also been proposed. These are powders which are dispersed on the water. They commonly incorporate an alcohol (such as cetyl or stearyl alcohol), which is known to help prevent evaporation by forming a barrier on the surface. An additive such as hydrated lime may also be blended with the alcohol to assist the alcohol to self-spread by creating charged particles.

The fundamental disadvantage with alcohol based monolayers is that the film typically degrades within a couples of days and therefore must be regularly re-applied to remain an effective barrier to water loss. In addition, trials have shown that alcohol layers are only partially successful in reducing evaporation with evaporation reductions of less than 30% reported.

Therefore there is a need for a cost effective composition for application to a liquid as a protective surface film which reduces the evaporation loss of the liquid for an extended period of time.

### SUMMARY OF THE INVENTION

In a first embodiment of the present invention there is provided a composition for protecting a liquid surface, the composition including a silicon based polymer.

The silicon based polymer is preferably a silicone based polymer which is known to be hydrophobic (water repellent) and possess a very silky feel which has led to their use in personal care products such as cosmetics, deodorants and shaving gels.

Silicones, or polysiloxanes, are inorganic-organic polymers with the chemical formula [R₂SiO]ₙ, where R=organic groups such as methyl, ethyl, and phenyl. These materials consist of an inorganic silicon-oxygen backbone ( . . . —Si—O—Si—O—Si−O— . . . ) with organic side groups attached to the silicon atoms, which are four-coordinate. In some cases organic side groups can be used to link two or more of these —Si—O— backbones together. By varying the —Si—O— chain lengths, side groups, and crosslinking, silicones can be synthesized with a wide variety of properties and compositions. They can vary in consistency from liquid to gel to rubber to hard plastic. The most common type is linear polydimethylsiloxane or PDMS.

The applicant has found that silicon based polymers (including silicone base polymers) can be advantageously employed as a large scale liquid surface protector, such as unenclosed water storage facilities (eg. reservoirs or dams). In particular, silicone base polymers possess a number of desirable properties, at least some of which are unexpected, including:
a) it is easy to apply (in liquid form), either from the banks of the water storage, by boat or by aerial spraying;
b) it floats naturally without the need for added buoyancy (the specific gravity is less than 1.0);
c) it tends to self-spread on the surface—even against significant wave pressure, such that is experienced on exposed bodies of water;
d) it has a high mechanical strength and even if the film is broken, it readily re-forms, thereby maintaining the integrity and functionality of the film;
e) it is resistant to high temperatures, oxidation and ultra violet breakdown;
f) it is transparent and has a high oxygen permeability and therefore has minimal impact upon sub-surface aquatic life.
g) it is classified as non-hazardous and can therefore be safely applied to drinking water bodies; and
h) it remains effective for at least several weeks.

The silicone polymer may be in a liquid or solid form, with the product dispersing into a film upon contact with the liquid surface. Preferably, the silicon polymer is a polyorganosiloxane, such as poly(dimethylsiloxane), which may have a variety of forms including fluids, elastomers and resins. Silicone polymer fluids are typically straight chain polymers of dimethylsiloxane. For ease of application the fluids preferably have a viscosity between 350 and 1000 centistokes.

By progressively increasing the cross linking of the dimethylsiloxane, solid elastomers and resins are formed. Silicone polymers with increasing levels of cross linking (and therefore solid) may be applied in a powder form. Dispersants are preferably added to the solid composition to aid in the uniform spreading of the polymer over the liquid surface. The higher cross linking in the solid compositions leads to a thicker polymer surface film which would be expected to provide a better heat and mass transfer barrier at the liquid/gas interface.

To assist in the identification of the spread of the composition on the liquid surface, a coloured dye or filler material (inert to the liquid to be protected) may be added. Examples of inert filler materials include calcium carbonate, talc, fine coal particulates, fly ash or cenospheres (a lightweight, inert, hollow sphere filled with inert air or gas, which may be derived from fly ash). This list is not exhaustive and other fillers may be used, with their selection based upon cost, density, hydrophobicity, inertness to the liquid to be protected and their ability to coat and adhere to the silicon based polymer.

The inert filler may also serve to improve the functionality of the composition by improving the dispersion and/or binding properties of the resultant film. Further, the filler may serve to reflect a portion of the radiant heat from the liquid/gas interface thereby lowering the partial pressure of the liquid above the liquid/gas interface, thus reducing the rate of evaporation of liquid through the protective film.

The filler material is preferably hydrophobic and of lower density than the liquid medium, such that the filler floats on the liquid surface. The particle size of the filler is typically between 2 and 50 microns.

The silicone based polymer film may be chemically modified to enhance adhesion of the film onto the filler material. In extreme environments, UV additives or other polymer stabilisers may also be added to the composition.

The composition preferably includes dimethyl siloxane in the range of 10 to 100 w/w %; a filler in the range of 0 to 90 w/w %; and a combined binder/dispersant in the range 0 to 10 w/w %. In compositions which include an inert filler, the weight ratio of the silicone polymer to filler is preferably between 1:10 to 1:1 and more preferably in the weight ratio of 1:7 to 1:4.

The dimethyl siloxane is preferably of a type similar to Dow Corning “200 Fluid” and has a viscosity of between 350 to 1000 centistokes.

In one preferred aspect of this first embodiment, the filler material may also possess other desirable functional properties. For instance, the surface film may also act as an insect suppressant and in particularly a mosquito suppressant. The dual functionality of the protective surface film addressing both water conservation and public health needs.

The suppressant may be a liquid or may form part of or be bound to the filler material. For example, sulphur and sulphur containing material are known to repel mosquitoes.

Therefore, coal particulates with sufficient sulphur content may be advantageously used.

In certain applications, particularly in more mild environments, organic polymers such as vegetable oils (e.g. canola, cottonseed, castor or linseed) may be blended with the silicone polymer to reduce the overall cost of the composition.

In a second embodiment of the present invention there is provided a method for protecting the surface of a liquid comprising the steps of:

applying a silicone based polymer composition to the liquid surface, wherein the silicone composition forms a substantially continuous protective film across the surface of the liquid.

The liquid surface area is preferably large, such as water storage facilities (eg. reservoirs or dams). The composition may be applied from the periphery of the liquid body or aerially, eg. via an airplane, such as a crop duster.

The continuous silicone based film preferably inhibits or controls the rate of heat and mass transfer across liquid/gas interface, such that evaporation of the liquid is reduced. The continuous silicone based polymer film may contain active ingredients which protect the liquid/gas interface from insect infestations.

For liquid compositions, the silicone based polymer composition is preferably applied at an application rate of about 0.3 to 50 grams per square meter, more preferably about 0.3 to 30 grams per square meter, and even more preferably 0.3 to 10 grams per square meter, and most preferably about 0.3 to 1.0 grams per square meter.

For solid compositions, the silicone based polymer composition is preferably applied at an application rate of about 1 to 200 grams per square meter, more preferably about 3 to 90 grams per square meter, and even more preferably 3 to 10 grams per square meter, and most preferably about 3 to 10 grams per square meter.

The relatively low application rates and high stability of the silicone based polymer compositions, compared to alcohol based films, enables a protective surface film to be applied in a cost effective manner. Further, as the composition is substantially inorganic, the price of the composition will remain relatively stable in comparison to petroleum based films.

For solid compositions, the method may also include the step of coating the filler with the silicone based polymer. This may be achieved through blending the filler under conditions which enable a uniform coating to be achieved over the surface of the filler. The required conditions may include the incorporation of additives such as binding agents and/or the application of heat to the blend.

It will be appreciated that the protective surface film may be applied to a range of liquid surfaces to inhibit or control mass and heat transfer from liquid/gas interface or form a physical and/or chemical barrier against the gaseous environment. For example, the silicone based polymer composition may be applied to an oxidizable liquid to prevent or reduce oxidation.

### EXAMPLES

Examples of the compositions were prepared by mixing the silicone based polymer, with insert fillers and other additives is described. Compositions in Examples 1, 2, 3 and a pure dimethyl siloxane solution were applied to a water surface in containers. The containers were then put in a commercial oven at 50° C. for a 12 hour period. The results indicated that the composition reduced the rate of evaporation by about 80-90% compared to a water sample with no protective film.

A protective surface film of dimethyl siloxane solution on a body of water exposed to the external environment was found to show no signs of degradation after a 4 week period. Given the inert nature of silicone based polymers, the protective films of the present invention would be expected maintain their functionality for a significantly longer period of time.

### Evaporation Retardant Formulations

### Example 1

| | | Indicative | Preferred | |
|---|---|---|---|---|
| Purpose | Component | Brand | Specification | w/w % |
| Inert filler | Fine dry carbon | na | Up to 20 | 75-85% |
| | | | microns | |
| Film forming | Dimethyl siloxane | Dow Corning | Viscosity | 12-17% |
| polymer | | “200 Fluid” | 350-1000 | |
| | | | centistokes | |
| Combined | Thermoplastic | Vinnapas | 524 Z | 3-8% |
| Dispersant/ | synthetic resin | | | |
| Binder | polyvinyl acetate | | | |

### Example 2

| | | Indicative | Preferred | |
|---|---|---|---|---|
| Purpose | Component | Brand | Specification | w/w % |
| Inert filler | Fly ash | na | Up to 2 | 70-80% |
| | | | microns | |
| Film forming | Dimethyl siloxane | Dow Corning | Viscosity | 15-22% |
| polymer | | “200 Fluid” | 350-1000 | |
| | | | centistokes | |
| Combined | Thermoplastic | Vinnapas | 524 Z | 5-10% |
| Dispersant/ | synthetic resin | | | |
| Binder | polyvinyl acetate | | | |

### Example 3

| | | Indicative | Preferred | |
|---|---|---|---|---|
| Purpose | Component | Brand | Specification | w/w % |
| Inert filler | Graphite | na | Up to 50 | 85-90% |
| | | | microns | |
| Film forming | Dimethyl siloxane | Dow Corning | Viscosity | 10-15% |
| polymer | | “200 Fluid” | 350-1000 | |
| | | | centistokes | |
| Combined | Thermoplastic | Vinnapas | 524 Z | 2-6% |
| Dispersant/ | synthetic resin | | | |
| Binder | polyvinyl acetate | | | |

### Mosquito Suppressant Formulations

### Example 4

| | | Indicative | Preferred | |
|---|---|---|---|---|
| Purpose | Component | Brand | Specification | w/w % |
| Active | elemental sulphur | na | Up to 20 | 85-90% |
| component | | | microns | |
| Film forming | Dimethyl siloxane | Dow Corning | Viscosity | 10-15% |
| polymer | | “200 Fluid” | 350-1000 | |
| | | | centistokes | |
| Combined | Thermoplastic | Vinnapas | 524 Z | 2-6% |
| Dispersant/ | synthetic resin | | | |
| Binder | polyvinyl acetate | | | |

### Example 5

| | | Indicative | Preferred | |
|---|---|---|---|---|
| Purpose | Component | Brand | Specification | w/w % |
| Active | Pyrethroid | Various | na | 0.1-0.2% |
| component | | | | |
| Film forming | Dimethyl | Dow Corning | Viscosity | 99.8-99.9% |
| polymer | siloxane | “200 Fluid” | 350-1000 | |
| | | | centistokes | |

### Comparative Evaporation Rate of Compositions of the Invention

| | | | | Dimethyl | |
|---|---|---|---|---|---|
| | Example | Example | Example | Siloxane | Control |
| | 1 | 2 | 3 | Liquid | (No film) |
| Application Rate | 5.0 | 5.0 | 5.0 | 0.5 | 0.0 |
| Equivalent | | | | | |
| (gm/sq.m.) | | | | | |
| Initial height in flask | 10.0 | 10.0 | 10.0 | 10.0 | 10.0 |
| Final height in flask | 9.5 | 9.2 | 9.2 | 9.7 | 6.5 |
| Drop | 0.5 | 0.8 | 0.8 | 0.3 | 3.5 |
| % Drop | 5% | 8% | 8% | 3% | 35% |

It will be understood that the invention disclosed and defined in this specification extends to all alternative combinations of two or more of the individual features mentioned or evident from the text. All of these different combinations constitute various alternative aspects of the invention.

1. A non-aqueous composition for the protection of a body of water comprising
5 to 95 wt % silicone polymer;
greater than zero percent to 90 wt % carrier material, wherein the carrier material is mineral oil or vegetable oil;
greater than zero to 20 wt % surfactant, wherein
a substantially continuous protective film is formed across a surface of the body of water when the composition is applied to the surface at a rate of 0.3 to 90 g/m².

2. The composition according to claim 1, wherein the polymer is a polyorganosiloxane.

3. The composition according to claim 2, wherein the polymer is a polymer of dimethylsiloxane.

4. The composition of claim 1, wherein the surfactant is present in an amount of 0.5 to 20 wt %.

5. The composition of claim 1, wherein the surfactant is present in an amount of 0.5 to 10 wt %.

6. The composition of claim 1, wherein the mineral oil or vegetable oil is present in the range of 5 to 90 wt %.

7. The composition of claim 1, wherein the mineral oil or vegetable oil is present in the range of 5 to 75 wt %.

8. The composition of claim 1, wherein the composition is a liquid at ambient temperature.

9. The composition of claim 1 having a viscosity of between 50 to 1000 centistokes at ambient temperature.

10. A method for protecting the surface of a liquid comprising the steps of:
applying a non-aqueous film forming composition to the liquid surface at a rate of 0.3 to 90 g/m², wherein the film forming composition comprises
5 to 95 wt % silicone polymer;
greater than zero percent to 90 wt % carrier material, wherein the carrier material is mineral oil or vegetable oil; and
greater than zero to 20 wt % surfactant,
The film forming composition forming a substantially continuous protective film across the surface of the liquid.

11. The method of claim 10 wherein the composition is a liquid composition.

12. The method of claim 10 wherein the surfactant is present in an amount of 0.2 to 20 wt %.

13. The method according to claim 10, wherein the carrier material is present in the range of 5 to 90 wt %.

14. The method of claim 11 or 12, wherein the surfactant and the carrier material are mixed prior to forming the film forming composition with the silicone polymer.

15. The method according to claim 10, wherein the continuous protective film protects the liquid/gas interface from insect infestations.

16. The method according to claim 10, wherein the composition is applied to the surface at a rate of between 0.3 to 50 g/m².
